(19) United States
(12) Patent Application Publication
(10) Pub. No.: US 2026/0141989 A1
(43) Pub. Date: May 21, 2026

(54) SYSTEM AND METHOD FOR MACHINE LEARNING ANALYSIS OF BIOTHERAPEUTICS

(71) Applicant: PURDUE RESEARCH FOUNDATION, West Lafayette, IN (US)

(72) Inventors: Arezoo Motavalizadeh Ardekani, West Lafayette, IN (US); Gaurav Kumar, West Lafayette, IN (US)

(21) Appl. No.: 19/388,514

(22) Filed: Nov. 13, 2025

Related U.S. Application Data
(60) Provisional application No. 63/721,615, filed on Nov. 18, 2024.
(60) Provisional application No. 63/791,883, filed on Apr. 21, 2025.

(51) Int. Cl. G16C 20/70; G06N 20/20; G16C 20/30; G16C 20/50

(52) U.S. Cl. CPC G16C 20/70; G06N 20/20; G16C 20/30; G16C 20/50

(57) ABSTRACT

Technologies for quantitative structure-activity relationship (QSAR) modeling for lipid nanoparticle (LNP) biotherapeutics include a computing device that receives a training data set including LNP test results, which each include an LNP chemical formulation and a corresponding result value of an LNP target variable, such as activity or cytotoxicity. The computing device extracts multiple input features for each LNP chemical formulation, where each of the input features is indicative of an attribute of a component or a composition of the LNP chemical formulation. The computing device trains a machine learning model to predict the LNP target variable with the input features and the result values of the training data set. A computing device may predict the LNP target variable result by extracting input features from a supplied LNP chemical formulation and supplying the input features to the trained machine learning model. Other embodiments are described and claimed.

### CROSS-REFERENCE TO RELATED APPLICATIONS

This application claims the benefit of and priority to U.S. Patent Application No. 63/721,615, entitled “SYSTEM AND METHOD FOR MACHINE LEARNING ANALYSIS OF BIOTHERAPEUTICS,” which was filed on Nov. 14, 2024, and to U.S. Patent Application No. 63/791,883, entitled “SYSTEM AND METHOD FOR MACHINE LEARNING ANALYSIS OF BIOTHERAPEUTICS,” which was filed on Apr. 21, 2025, each of which is incorporated herein by reference in its entirety.

### BACKGROUND

Lipid nanoparticles (LNPs) are highly effective carriers for gene therapies, including mRNA and siRNA delivery, due to their ability to transport nucleic acids across biological membranes, low cytotoxicity, improved pharmacokinetics, and scalability. These nanoparticles are engineered to encapsulate and transport nucleic acids, ensuring their safe and efficient transport into cells. Owing to their size and properties, LNPs are taken up by cells via endocytosis. The ionizability of the lipids at low pH is believed to facilitate endosomal escape, releasing the cargo into the cytoplasm where mRNA can be translated into proteins or siRNA can induce gene silencing. LNPs designed for nucleic acid delivery typically include four constituents: (1) ionizable lipids to encapsulate nucleic acids and facilitate endosomal escape, (2) helper lipids, (3) cholesterol to enhance membrane rigidity, ensure particle stability, and improve intracellular delivery, and (4) a polyethylene glycol (PEG)-lipid conjugate to extend the circulation time of LNPs by preventing rapid clearance. The chemical structures of LNP components can have a significant impact on transfection efficacy and organ selectivity.

A typical approach to formulate LNPs is to establish a quantitative structure-activity relationship (QSAR) between their compositions and in vitro/in vivo activities, which allows for the prediction of activity based on molecular structure. Typical QSAR for LNPs has limited predictive performance due to the complexity of multi-component formulations, interactions with biological membranes, stability in physiological environments, and diverse physicochemical properties.

### SUMMARY

According to one aspect of the disclosure, a computing device for quantitative structure-activity relationship model training includes a training data manager, a featurizer, and a model trainer. The training data manager is configured to receive a first training data set comprising a plurality of lipid nanoparticle (LNP) test results. Each of the LNP test results comprises an LNP chemical formulation and a corresponding result value for an LNP target variable. The featurizer is configured to extract a plurality of input features for each LNP chemical formulation of the plurality of LNP test results. Each of the input features is indicative of an attribute of a component of the LNP chemical formulation or a composition of the LNP chemical formulation. The model trainer is configured to train a machine learning model to predict the LNP target variable with the plurality of input features and the corresponding result values of the LNP test results.

In an embodiment, the LNP target variable comprises LNP activity or cytotoxicity. In an embodiment, each LNP chemical formulation of the LNP test results is indicative of a plurality of components of an LNP, including an ionizable lipid component, a helper lipid component, a PEG-lipid component, a cholesterol component, and a nucleic acid component.

In an embodiment, the computing device further includes a data preprocessor configured to preprocess the corresponding result values for the LNP target variables of the first training data set to generate processed result values. The machine learning model is trained with the plurality of input features and the processed result values. To preprocess the corresponding result values includes to log-transform any numeric result values of the corresponding result values to generate log-normalized numeric result values, to normalize the log-transformed numeric result values to generate normalized numeric result values, and to categorize the normalized numeric result values with k predetermined categorical labels using k-means clustering.

In an embodiment, the machine learning model comprises a decision tree ensemble model. In an embodiment, the decision tree ensemble model comprises a balanced random forest model or an extra trees model.

In an embodiment, to extract the plurality of input features for each LNP chemical formulation of the plurality of LNP test results includes, for a first LNP chemical formulation of the plurality of LNP test results, to determine a chemical structure of each component of the first LNP chemical formulation, to extract a plurality of molecular structural features in response to a determination of the chemical structure of the first LNP chemical formulation, and to enrich the plurality of molecular structural features with a plurality of composition-level features associated with the first LNP chemical formulation. In an embodiment, to extract the plurality of molecular structural features includes to encode a plurality of quantitative molecular descriptors into a numerical vector for each component of the first LNP chemical formulation, and to concatenate the numerical vectors for each component of the first LNP chemical formulation. In an embodiment, to enrich the plurality of molecular structural features with the plurality of composition-level features includes to enrich the plurality of molecular structural features with a molar ratio of a plurality of components of the first LNP chemical formulation, an encapsulated nucleic acid type of the first LNP chemical formulation, a ratio of lipids to nucleic acid of the first LNP chemical formulation, or a drug dosage associated with the first LNP chemical formulation. In an embodiment, to extract the plurality of molecular structural features further includes to normalize the plurality of molecular structural features.

In an embodiment, the model trainer is further configured to perform transfer learning with the machine learning model based on a second trained machine learning model. The first training data set comprises an in vivo data set, and the second trained machine learning model is trained on an in vitro training data set.

According to another aspect, a method for quantitative structure-activity relationship model training includes receiving, by a computing device, a first training data set comprising a plurality of lipid nanoparticle (LNP) test results, wherein each of the LNP test results comprises an LNP chemical formulation and a corresponding result value for an LNP target variable; extracting, by the computing device, a plurality of input features for each LNP chemical formulation of the plurality of LNP test results, wherein each of the input features is indicative of an attribute of a component of the LNP chemical formulation or a composition of the LNP chemical formulation; and training, by the computing device, a machine learning model to predict the LNP target variable using the plurality of input features and the corresponding result values of the LNP test results.

In an embodiment, the method further includes preprocessing, by the computing device, the corresponding result values for the LNP target variables of the first training data set to generate processed result values, wherein preprocessing the corresponding result values includes log-transforming any numeric result values of the corresponding result values to generate log-normalized numeric result values; normalizing the log-transformed numeric result values to generate normalized numeric result values; and categorizing the normalized numeric result values with k predetermined categorical labels using k-means clustering; wherein training the machine learning model includes training the machine learning model using the plurality of input features and the processed result values.

In an embodiment, extracting the plurality of input features for each LNP chemical formulation of the plurality of LNP test results comprises, for a first LNP chemical formulation of the plurality of LNP test results determining a chemical structure of each component of the first LNP chemical formulation; extracting a plurality of molecular structural features in response to determining the chemical structure of the first LNP chemical formulation; and enriching the plurality of molecular structural features with a plurality of composition-level features associated with the first LNP chemical formulation. In an embodiment, extracting the plurality of molecular structural features comprises encoding a plurality of quantitative molecular descriptors into a numerical vector for each component of the first LNP chemical formulation; and concatenating the numerical vectors for each component of the first LNP chemical formulation.

In an embodiment, the method further includes performing, by the computing device, transfer learning with the machine learning model based on a second trained machine learning model, wherein the first training data set comprises an in vivo data set, and wherein the second trained machine learning model is trained on an in vitro training data set.

According to another aspect, a computing device for quantitative structure-activity relationship estimation includes an inference data manager, a featurizer, and a prediction engine. The inference data manager is configured to receive a first lipid nanoparticle (LNP) chemical formulation. The featurizer is configured to extract a plurality of input features for the first LNP chemical formulation. Each of the input features is indicative of an attribute of a component of the first LNP chemical formulation or a composition of the first LNP chemical formulation. The prediction engine is configured to predict an LNP target variable result for the first LNP chemical formulation with a trained machine learning model by providing the plurality of input features to the trained machine learning model. The target variable result comprises LNP activity or cytotoxicity.

In an embodiment, the trained machine learning model comprises a decision tree ensemble model. The decision tree ensemble model comprises a balanced random forest model or an extra trees model.

In an embodiment, to extract the plurality of input features for the first LNP chemical formulation includes to determine a chemical structure of each component of the first LNP chemical formulation, to extract a plurality of molecular structural features in response to determining the chemical structure of the first LNP chemical formulation, and to enrich the plurality of molecular structural features with a plurality of composition-level features associated with the first LNP chemical formulation. In an embodiment, to extract the plurality of molecular structural features includes to encode a plurality of quantitative molecular descriptors into a numerical vector for each component of the first LNP chemical formulation, and to concatenate the numerical vectors for each component of the first LNP chemical formulation.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a system for quantitative structure-activity relationship modeling for lipid nanoparticle biotherapeutics;

FIG. 2 is a simplified block diagram of at least one embodiment of an environment that may be established by a computing device of the system of FIG. 1;

FIG. 3 is a simplified flow diagram of at least one embodiment of a method for training a machine learning model for quantitative structure-activity relationship prediction for lipid nanoparticle biotherapeutics that may be executed by the system of FIGS. 1 and 2;

FIG. 4 is a simplified flow diagram of at least one embodiment of a method for input feature extraction that may be executed in connection with the method of FIG. 3;

FIG. 5 is a simplified flow diagram of at least one embodiment of a method for quantitative structure-activity relationship prediction for lipid nanoparticle biotherapeutics that may be executed by the system of FIGS. 1 and 2;

FIG. 6 is a simplified flow diagram of at least one embodiment of a method for multi-phase feature refinement that may be executed by the system of FIGS. 1 and 2; and

FIGS. 7-12 are diagrams of charts illustrating experimental results that were achieved by a system according to FIGS. 1 and 2.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

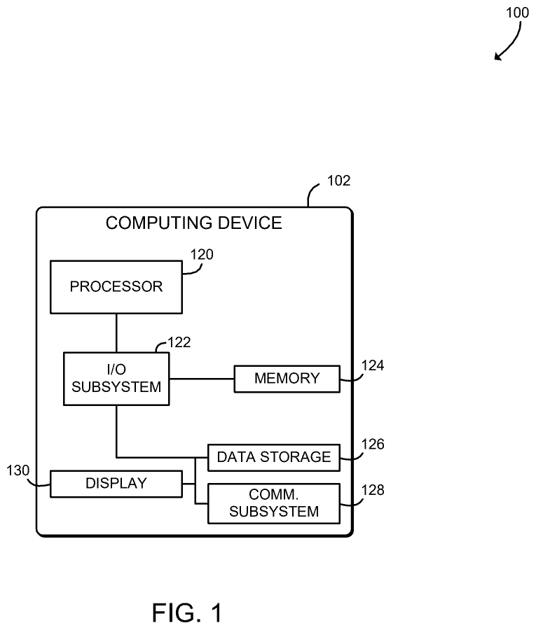

Referring now to FIG. 1, an illustrative system 100 for quantitative structure-activity relationship (QSAR) modeling for lipid nanoparticle (LNP) biotherapeutics includes a computing device 102. In use, the computing device 102 collects and processes a large training data set that relates lipid nanoparticle (LNP) chemical formulations to target variable results such as LNP activity or cytotoxicity. The training data set may originate from in vitro and/or in vivo studies. The computing device 102 extracts features from the LNP chemical formulation that are indicative of the chemical structure of the LNP as well as composition-level features of the LNP formulation. The computing device 102 trains a machine learning model, such as a decision tree ensemble model, with the extracted input features from the training data. The computing device 102 may combine training from smaller datasets (e.g., in vivo studies) with training from larger datasets (e.g., in vitro studies) using transfer learning. The computing device 102 (or another device) may use such a trained machine learning model to predict target variable values (e.g., LNP activity and/or cytotoxicity) for new LNP formulations. In the illustrative system 100, the machine learning model is trained on a much larger number of LNP formulations as compared to existing systems, which improves prediction performance, for example by reducing overfitting and otherwise improving accuracy when applied to a diverse set of LNPs. Additionally, the system 100 trains the machine learning model on composition-level features such as molar ratios and drug dosage, which improves prediction performance compared to existing systems, which typically do not train on composition-level features. Additionally, and as described further below, an improvement in accuracy was realized by identifying LNP features and other features that exhibit strong interactions and/or dependencies with the LNP features using feature analysis.

The computing device 102 may be embodied as any type of device capable of performing the functions described herein. For example, the computing device 102 may be embodied as, without limitation, a workstation, a desktop computer, a laptop computer, a server, a rack-mounted server, a blade server, a network appliance, a web appliance, a tablet computer, a smartphone, a consumer electronic device, a distributed computing system, a multiprocessor system, and/or any other computing device capable of performing the functions described herein. Additionally, in some embodiments, the computing device 102 may be embodied as a “virtual server” formed from multiple computing devices distributed across a network and operating in a public or private cloud. Accordingly, although the computing device 102 is illustrated in FIG. 1 as embodied as a single computing device, it should be appreciated that the computing device 102 may be embodied as multiple devices cooperating together to facilitate the functionality described below. As shown in FIG. 1, the illustrative computing device 102 includes a processor 120, an I/O subsystem 122, memory 124, a data storage device 126, and communication circuitry 128. Of course, the computing device 102 may include other or additional components, such as those commonly found in a workstation computer (e.g., various input/output devices), in other embodiments. Additionally, in some embodiments, one or more of the illustrative components may be incorporated in, or otherwise form a portion of, another component. For example, the memory 124, or portions thereof, may be incorporated in the processor 120 in some embodiments.

The processor 120 may be embodied as any type of processor or compute engine capable of performing the functions described herein. For example, the processor may be embodied as a single or multi-core processor(s), digital signal processor, microcontroller, or other processor or processing/controlling circuit. Similarly, the memory 124 may be embodied as any type of volatile or non-volatile memory or data storage capable of performing the functions described herein. In operation, the memory 124 may store various data and software used during operation of the computing device 102 such as operating systems, applications, programs, libraries, and drivers. The memory 124 is communicatively coupled to the processor 120 via the I/O subsystem 122, which may be embodied as circuitry and/or components to facilitate input/output operations with the processor 120, the memory 124, and other components of the computing device 102. For example, the I/O subsystem 122 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 122 may form a portion of a system-on-a-chip (SoC) and be incorporated, along with the processor 120, the memory 124, and other components of the computing device 102, on a single integrated circuit chip.

The data storage device 126 may be embodied as any type of device or devices configured for short-term or long-term storage of data such as, for example, memory devices and circuits, memory cards, hard disk drives, solid-state drives, or other data storage devices. The communication circuitry 128 of the computing device 102 may be embodied as any communication circuit, device, or collection thereof, capable of enabling communications between the computing device 102 and remote devices. The communication circuitry 128 may be configured to use any one or more communication technology (e.g., wireless or wired communications) and associated protocols (e.g., Ethernet, Bluetooth®, Wi-Fi®, WiMAX, etc.) to effect such communication.

As shown in FIG. 1, the computing device 102 may include a display 130. The display 130 may be embodied as any type of display capable of displaying digital images or other information, such as a liquid crystal display (LCD), a light emitting diode (LED), a plasma display, a cathode ray tube (CRT), or other type of display device. In some embodiments, the display 130 may be coupled to a touch screen to allow user interaction with the computing device 102.

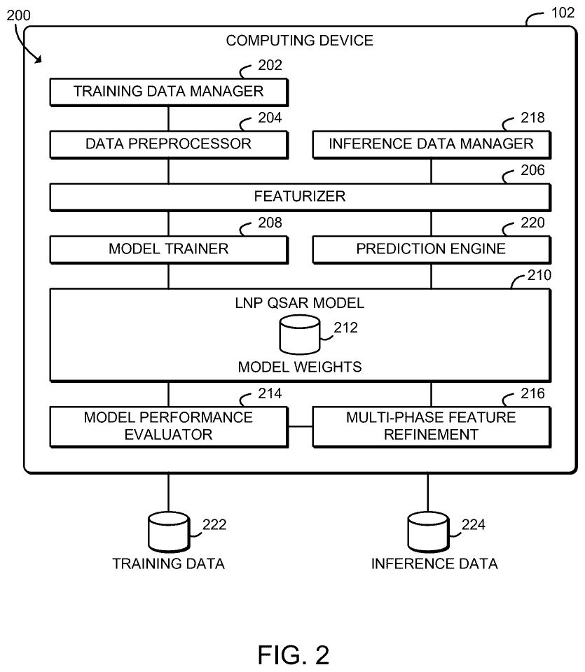

Referring now to FIG. 2, in the illustrative embodiment, the computing device 102 establishes an environment 200 during operation. The illustrative environment 200 includes a training data manager 202, a data preprocessor 204, a featurizer 206, a model trainer 208, a model performance evaluator 214, multi-phase feature refinement 216, an inference data manager 218, and a prediction engine 220. The various components of the environment 200 may be embodied as hardware, firmware, software, or a combination thereof. As such, in some embodiments, one or more of the components of the environment 200 may be embodied as circuitry or a collection of electrical devices (e.g., training data manager circuitry 202, data preprocessor circuitry 204, featurizer circuitry 206, model trainer circuitry 208, model performance evaluator circuitry 214, multi-phase feature refinement circuitry 216, inference data manager circuitry 218, and/or prediction engine circuitry 220). It should be appreciated that, in such embodiments, one or more of those components may form a portion of the processor 120, the I/O subsystem 122, and/or other components of the computing device 102.

The training data manager 202 is configured to receive a training data set 222 that includes multiple lipid nanoparticle (LNP) test results. Each of those LNP test results includes an LNP chemical formulation and a corresponding result value for an LNP target variable. The LNP target variable may include LNP activity (e.g., gene expression, protein activity, etc.) or cytotoxicity. The LNP chemical formulation is indicative of multiple components of an LNP, including an ionizable lipid component, a helper lipid component, a PEG-lipid component, a cholesterol component, and a nucleic acid component. In some embodiments, the training data manager 202 further collects metadata associated with the LNP test results.

The data preprocessor 204 is configured to preprocess the corresponding result values for the LNP target variables of the training data set 222 to generate processed result values. Preprocessing the result values may include log-transforming numeric result values to generate log-normalized numeric result values, normalizing the log-transformed numeric result values to generate normalized numeric result values, and categorizing the normalized numeric result values with k predetermined categorical labels using k-means clustering. In some embodiments, the data preprocessor 204 is further configured to preprocess the training data set 222 by converting names of lipid molecules to SMILES (Simplified Molecular Input Line Entry System) representations, and by one-hot encoding experimental variables.

The featurizer 206 is configured to extract input features for each LNP chemical formulation of the LNP test results. Each of those input features is indicative of an attribute of a component of the LNP chemical formulation or a composition of the LNP chemical formulation. Extracting the input features may include, for each LNP chemical formulation, determining a chemical structure of each component of the LNP chemical formulation, extracting molecular structural features in response to determining the chemical structure, and enriching the molecular structural features with composition-level features associated with the LNP chemical formulation. Extracting the molecular structural features may include encoding quantitative molecular descriptors into a numerical vector for each component of the LNP chemical formulation and concatenating the numerical vectors for each component of the LNP chemical formulation. The composition-level features may include a molar ratio of components of the LNP chemical formulation, an encapsulated nucleic acid type of the LNP chemical formulation, a ratio of lipids to nucleic acid of the LNP chemical formulation, or a drug dosage associated with the LNP chemical formulation. In some embodiments, extracting the molecular structural features may include normalizing the molecular structural features.

The model trainer 208 is configured to train an LNP QSAR machine learning model 210 to predict the LNP target variable using the input features and the corresponding result values of the LNP test results. In some embodiments, the machine learning model 210 may be trained with the input features and the processed result values. Training the machine learning model 210 may further include performing multi-fold validation. In some embodiments, the model trainer 208 is further configured to perform transfer learning with the machine learning model 210 based on a pretrained machine learning model 210. In some embodiments, the training data set 222 may be an in vivo data set, and the pretrained machine learning model 210 may be trained on an in vitro training data set.

The LNP QSAR model 210 is a machine learning model, and in the illustrative embodiment is a decision tree ensemble model. For example, in some embodiments, the decision tree ensemble model may be embodied as a balanced random forest model or an extra trees model. In other embodiments, the model 210 may be embodied as a support vector machine (SVM), artificial neural network (ANN), or any other machine learning classification model suitable for deep learning model suitable for classification tasks as described herein. Accordingly, the model 210 includes multiple weights 212, which are adjusted during model training.

The model performance evaluator 214 is configured to evaluate performance of the machine learning model 210 after training. In some embodiments, the model performance evaluator 214 may identify important features in the input features using an explainable model technique such as Shapley additive explanations (SHAP).

The multi-phase feature refinement circuitry 216 is configured to successively refine the set of input features to a minimal set of features that predict LNP activity with high fidelity. The multi-phase feature refinement circuitry 216 performs coarse refining by pruning features according to feature importance determined with the model performance evaluator 214. The multi-phase feature refinement circuitry 216 performs further refinement by mapping features to chemically interpretable feature classes, and by evaluating feature-level combinations constrained by prior refinements.

The inference data manager 218 is configured to receive an inference data set 224, which includes one or more lipid nanoparticle (LNP) chemical formulations. As described above, each LNP chemical formulation is indicative of a plurality of components including an ionizable lipid component, a helper lipid component, a PEG-lipid component, a cholesterol component, and a nucleic acid component.

The prediction engine 220 is configured to predict an LNP target variable result for the LNP chemical formulation of the inference data set 224 using the trained machine learning model 210 by providing multiple input features extracted from the LNP chemical formulation to the trained machine learning model. As described above, the target variable result may be LNP activity or cytotoxicity. As described above, the input features are extracted by the featurizer 206.

Although FIG. 2 illustrates the environment 200 as including all of the training data manager 202, the data preprocessor 204, the featurizer 206, the model trainer 208, the machine learning model 210, the model performance evaluator 214, the inference data manager 218, and the prediction engine 210, it should be understood that in some embodiments one or more of those components may be established by different computing devices 102, and the operations of those components may be distributed in space and/or in time. For example, in an embodiment the training data manager 202, the data preprocessor 204, the featurizer 206, and the model trainer 208 may be established by a computing device 102 configured to perform model training, for example in a cloud computing environment. After training, the trained LNP QSAR model 210 may be distributed to one or more additional computing devices 102. Continuing that example, the inference data manager 218, the featurizer 206, and the prediction engine 220 may be established by a computing device 102 configured to perform model inference, for example on a workstation device. Continuing that example further, the LNP QSAR model 210 may be established by a separate computing device 102 to perform inference, for example in a cloud computing environment. Of course, other configurations of computing devices are possible in other environments.

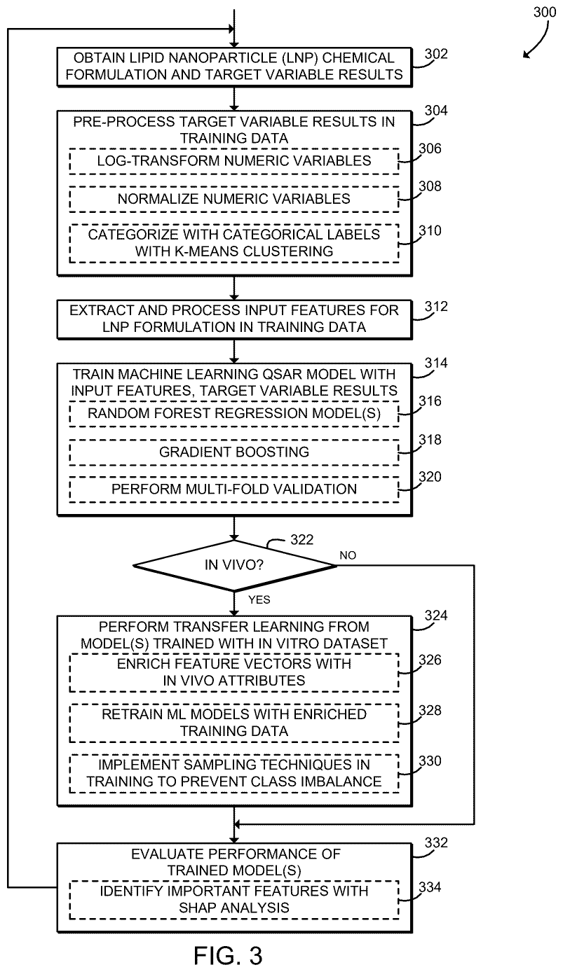

Referring now to FIG. 3, in use, the computing device 102 may execute a method 300 for training the LNP QSAR model 210. It should be appreciated that, in some embodiments, the operations of the method 300 may be performed by one or more components of the environment 200 of the computing device 102 as shown in FIG. 2. The method 300 begins with block 302, in which the computing device 102 obtains a training data set 222 including lipid nanoparticle (LNP) chemical formulations and associated target variable results.

The LNP chemical formulation describes the chemical structures and composition of the components of the LNP. The chemical structure of constituent molecules may be described using chemical structure drawings, programmatic representations such as simplified molecular-input line-entry system (SMILES) strings, or other representations. In an illustrative example, the LNP chemical formulation includes molar compositions of the components of the LNP, including an ionizable lipid, a helper lipid, a PEG-lipid, and cholesterol, as well as the type of encapsulated nucleic acid (illustratively mRNA or siRNA), lipid-to-nucleic acid ratios (wt/wt), and drug dosages (ng). The target variable results may include activity values that indicate the amount of mRNA translated into proteins or the extent of siRNA-induced gene silencing, which represents the efficacy of nucleic acid delivery by LNPs.

In block 304, the computing device 102 preprocesses the target variable results in the training data set 222. Due to heterogeneity in experimental conditions, measurement methods, and reporting formats, reported target variable values may vary across studies. Accordingly, the computing device 102 may preprocess the target variable results to standardize reported values across studies. In some embodiments, in block 306 the computing device 102 log-transforms numeric variables. Log-transformation may be applied before normalization to reduce skewness and stabilize variance in the distribution of target values, thereby ensuring more consistent scaling across datasets with differing measurement ranges. In some embodiments, in block 308 the computing device 102 normalizes numeric variables (including log-transformed numeric variables). In an embodiment, the computing device 102 performs min-max normalization within a dataset according to Equation 1, below, where the index i represents a dataset t_{j,i} denotes the log-transformed target value for LNP formulation j from dataset i, and ti,min and ti,max represent the minimum and maximum log-transformed values within dataset i. Normalization may ensure that target variable values range between 0 and 1.

$${t}_{j , i}^{n} = \frac{{t}_{j , i} - {t}_{i , min}}{{t}_{i , max} - {t}_{i , min}} \qquad ( 1 )$$

In some embodiments, in block 310 the computing device 102 categorizes numeric variables (including normalized variables) with a predetermined set of categorical labels, for example using a k-means clustering algorithm. For example, for binary classification, normalized target variables may be converted into “high” and “low” categorical labels. Continuing that example, for multiclass classification, normalized target variables may be converted into “high,” “mid-high,” “mid-low,” and “low” categorical labels, or another labeling scheme. For studies reporting target values in ranges, the highest reported range may be labeled as “high” and the remaining ranges as “low” (for binary classification), or the top three highest ranges labels may be assigned as “high,” “mid-high,” and “mid-low” (for multiclass classification).

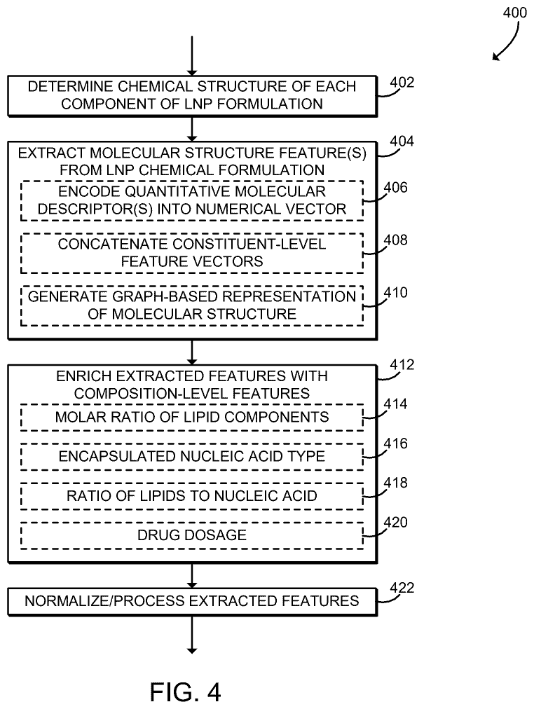

In block 312, the computing device 102 extracts and processes input features for the LNP formulations of the training data set 222. For example, the computing device 102 may extract a vector of input features for each LNP formulation included in the training data set 222. Each of the input features may be indicative of a chemical structural of one or more components of the LNP formulation, a composition-level feature of the LNP formulation, or an other attribute of the LNP formulation. One potential embodiment of a method for extracting and processing input features from an LNP formulation is described below in connection with FIG. 4.

In block 314, the computing device 102 trains the LNP QSAR model 210 with the extracted input features and the associated target variable results from the training data 222. The model 210 may be trained with part or all of the training data 222. For example, in an illustrative embodiment, part of the training data 222 was used for hyperparameter tuning to optimize machine learning model parameters, and the remaining training data 222 was used for model training. As another example, part of the training data 222 (or an independent training dataset 222) may be reserved for model validation. As described above, the LNP QSAR model 210 may be embodied as any appropriate machine learning classification model, including a decision tree ensemble model, support vector machines (SVM), artificial neural network (ANN), or other model. In some embodiments, in block 316 the computing device 102 trains a random forest regression model. In some embodiments, in block 318 the computing device 102 trains a gradient boosting model.

In some embodiments, in block 320 the computing device 102 performs multi-fold validation. For example, in an embodiment the computing device 102 may perform a scaffold-based 5-fold cross-validation. Continuing that example, the computing device 102 may generate a molecular scaffold from the SMILES representation (or other chemical structure representation) of the ionizable lipid and use that scaffold to perform splits in the training data. This scaffold-based approach may ensure minimal scaffold overlap between training and testing sets, simulating realistic scenarios wherein models must generalize to structurally distinct chemical entities.

In block 322, the computing device 102 determines whether the training data set 222 is an in vivo training data set, as opposed to an in vitro data set. That is, the computing device 102 may determine whether the training data set 222 is generated from in vivo studies as opposed to in vitro studies. If not, the method 300 branches ahead to block 332, described below. If the training data set 222 is an in vivo data set, the method 300 advances to block 324.

In block 324, the computing device 102 performs transfer learning from one or more models 210 that were previously trained using an in vitro dataset. As described above, the in vitro dataset may include more results or otherwise include more data than a comparable in vivo dataset. Accordingly, transfer learning leverages the knowledge learned from the larger in vitro dataset to improve predictive performance on the smaller in vivo dataset, where data availability is typically limited. In some embodiments, in block 326 the computing device 102 enriches input feature vectors with one or more in vivo attributes. In vivo attributes may include formulation properties relevant to biological performance of the LNP in vivo, such as particle size, polydispersity index (PDI), and zeta potential.

In some embodiments, in block 328 the computing device 102 retrains the models 210 with the enriched training data. For example, in an embodiment, the previously trained model 210 may be used to generate prediction probabilities for each LNP in the in vivo dataset. For each ML model and featurization technique, predicted probabilities corresponding to the “high” activity class were computed using the trained base models. Since each base model has been trained using scaffold-based cross-validation, the final prediction score for each LNP was computed as the average probability across the folds. This ensemble approach may ensure that the model predictions integrate information learned from multiple training subsets, enhancing robustness. The predicted probabilities are then combined with additional physicochemical properties, for example, particle size, PDI, and zeta potential, to construct an enriched feature set for each LNP. This enriched feature representation thus captures both the model-derived knowledge from in vitro training (through predicted probabilities from the pre-trained model 210) and new, biologically relevant in vivo attributes. Model training and evaluation on the in vivo dataset may be performed using scaffold-based n-fold cross-validation, analogous to the procedure followed for the in vitro dataset. This ensures consistency in model evaluation and allows for an assessment of the generalizability of knowledge transferred from in vitro to in vivo conditions. This transfer learning approach enables the incorporation of insights learned from a well-annotated, high-volume in vitro dataset into a distinct in vivo context, where data may be limited but additional biologically meaningful features are available.

In some embodiments, in block 330 the computing device 102 implements a sampling technique during training to prevent class imbalance. Class imbalance is a common challenge in biological datasets, as most studies tend to select only the top-performing LNP formulations from in vitro screens for subsequent in vivo evaluations. This selective sampling increases the likelihood of an overrepresentation of high-activity LNPs, which can bias ML models and compromise generalizability. Accordingly, the computing device 102 performs an oversampling technique such as Borderline SMOTE (Synthetic Minority Over-sampling Technique) and/or ADASYN (Adaptive Synthetic Sampling) on the enriched in vivo feature set to achieve a more balanced class distribution prior to model fitting.

In block 332, after performing training and/or transfer learning, the computing device 102 evaluates performance of the trained LNP QSAR model 210. The computing device 102 may be assessed using any appropriate metric suitable for binary and/or multiclass classification. In some embodiments, in block 334 the computing device 102 identifies important input features with an attribution analysis or other explainable model technique, such as Shapley additive explanations (SHAP). That is, the computing device 102 may identify which molecular and compositional features most strongly influence LNP activity. In some embodiments, in response to or otherwise in connection with identifying the important input features, the computing device 102 may perform multi-phase feature refinement to find a smaller group of features that are necessary and sufficient to predict LNP activity with high fidelity. One such method for feature refinement is described below in connection with FIG. 6. After training and evaluating performance of the model 210, the method 300 loops back to block 302, in which the computing device 102 may continue to collect additional training data 222 and perform additional training.

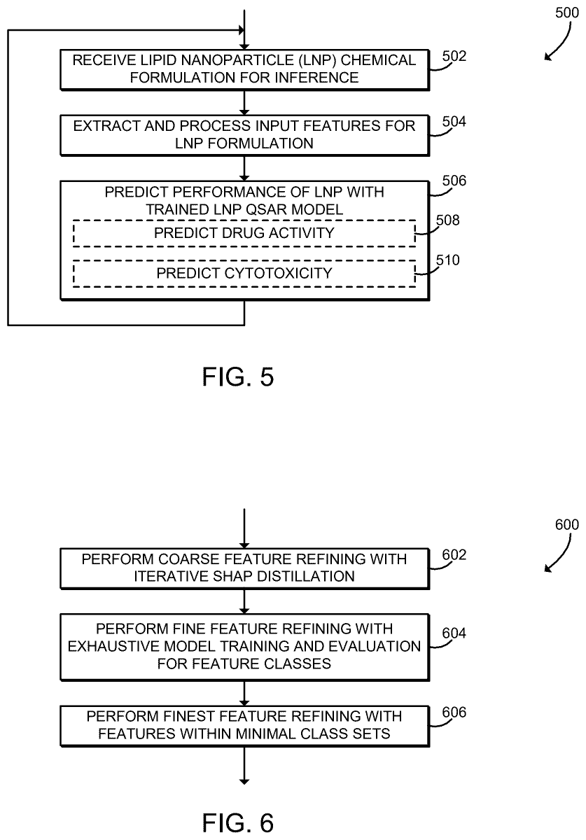

Referring now to FIG. 4, in use, the computing device 102 may execute a method 400 for input feature extraction. The method 400 may be executed in connection with block 312 of the method 300 shown in FIG. 3 and described above, and/or in connection with block 504 of the method 500 shown in FIG. 5 and described below. It should be appreciated that, in some embodiments, the operations of the method 400 may be performed by one or more components of the environment 200 of the computing device 102 as shown in FIG. 2. The method 400 begins with block 402, in which the computing device 102 determines a chemical structure of each component of an LNP formulation.

In block 404, the computing device 102 extracts one or more chemical structure features from the LNP chemical formulation. There are several types of featurization techniques, each capturing different aspects of molecular properties. For instance, molecular descriptors quantify properties such as molecular weight, partial charge, partition coefficient, and the number of hydrogen bond donors. Fingerprints represent the presence or absence of specific substructures or patterns within a molecule using binary vectors. Molecular graphs treat molecules as graphs, where atoms are nodes and bonds are edges, allowing the use of graph-based algorithms and neural networks to capture complex relationships and interactions within the molecular structure. In some embodiments, in block 406 the computing device 102 encodes one or more quantitative molecular descriptors based on the chemical structure of the component into a numerical vector. In some embodiments, in block 408 the computing device 102 concatenates numerical vectors from multiple components of the LNP formulation into a single feature vector. In some embodiments, in block 410 the computing device 102 generates a graph-based representation of the chemical structure of the component of the LNP formulation.

In block 412, the computing device 102 enriches the extracted features with one or more composition-level features, which are indicative of attributes of the LNP formulation composition as a whole rather than individual components. In some embodiments, in block 414 the computing device 102 enriches the extracted features with a molar ratio of lipid components of the LNP formulation. In some embodiments, in block 416 the computing device 102 enriches the extracted features with a type of the nucleic acid encapsulated in the LNP (e.g., mRNA, siRNA, DNA, or other nucleic acid). In some embodiments, in block 418 the computing device 102 enriches the extracted features with a ratio of lipids to nucleic acid in the LNP formulation. In some embodiments, in block 420 the computing device 102 enriches the extracted features with a drug dosage associated with the LNP formulation (e.g., dosage in ng, etc.).

In block 422, the computing device 102 normalizes or otherwise processes the extracted features. After processing, the method 400 is completed. The vector of extracted features may be subsequently used for model training as described above in connection with FIG. 3 and/or in connection with model inference/prediction as described below in connection with FIG. 5.

Referring now to FIG. 5, in use, the computing device 102 may execute a method 500 for activity prediction for LNP biotherapeutics using the trained LNP QSAR model 210. It should be appreciated that, in some embodiments, the operations of the method 500 may be performed by one or more components of the environment 200 of the computing device 102 as shown in FIG. 2. The method 500 begins with block 502, in which the computing device 102 receives an inference data set 224 including one or more lipid nanoparticle (LNP) chemical formulations for inference.

In block 504, the computing device 102 extracts and processes input features for the LNP formulations of the inference data set 224. The computing device 102 may perform similar or the same feature extraction as performed during model training and as described above in connection with block 312 of FIG. 3. For example, the computing device 102 may extract a vector of input features for each LNP formulation included in the inference data set 224. Each of the input features may be indicative of a chemical structural of one or more components of the LNP formulation, a composition-level feature of the LNP formulation, or an other attribute of the LNP formulation. One potential embodiment of a method for extracting and processing input features from an LNP formulation is described above in connection with FIG. 4.

In block 506, the computing device 102 predicts performance of the LNP described in the inference data set 224 using a trained LNP QSAR model 210. The LNP QSAR model 210 may be trained as described above in connection with FIG. 3. Based on the extracted input features provided to the LNP QSAR model 210, the LNP QSAR model 210 outputs one or more predictions of an LNP target variable. In some embodiments, in block 508 the computing device 102 predicts drug activity for the LNP formulation using the model 210. In some embodiments, in block 510 the computing device 102 predicts cytotoxicity for the LNP formulation using the model 210. After performing predictions with the trained model 210, the method 500 loops back to block 502, in which the computing device 102 may receive additional inference data 224 and perform additional predictions.

Referring now to FIG. 6, in use, the computing device 102 may execute a method 600 for multi-phase feature refinement after training LNP QSAR model 210. The method 600 may be executed in connection with block 332 of the method 300, as described above in connection with FIG. 3. It should be appreciated that, in some embodiments, the operations of the method 600 may be performed by one or more components of the environment 200 of the computing device 102 as shown in FIG. 2.

As described above, modern lipid nanoparticles (LNPs) may be described by hundreds of cheminformatic descriptors and formulation variables, which may be collectively referred to as features or input features. In practice, those descriptors may be conflated—that is, a single descriptor often encodes multiple physicochemical/biological properties, while the same underlying property is redundantly represented across many descriptors. In such a regime, conventional feature ranking reveals correlates of performance but rarely isolates the truly necessary and sufficient property sets that govern activity. Additionally, the naive path to certainty, testing all descriptor subsets, is often computationally impossible. For example, in an illustrative embodiment with K=895 descriptors, the hypothesis space is 2^{K} (i.e., >10²⁶⁹) potential models.

Accordingly, the illustrative system determines which minimal sets of descriptors, and, by implication, which minimal sets of interpretable property classes, are necessary and sufficient to predict LNP activity with high fidelity, and further illustrates how they can be discovered without brute-force enumeration. The illustrative technique explicitly anticipates non-uniqueness: e.g., multiple “minimal” sets of features may exist that are functionally indistinguishable (within a tolerance) in predictive performance.

As described further below, the system 100 implements a multi-phase refinement framework that converts an intractable descriptor search into a tractable and interpretable property-class search, while preserving predictive power and exposing mechanism-aligned variables. Together, the illustrative phases deliver computational tractability, interpretability via property classes, and robustness by acknowledging and enumerating equivalent minimal solutions rather than a single brittle optimum.

Additionally, the system 100 introduces a predictive sufficiency curve and associated selection rule that choose an iteration where model performance is maximized (or stabilized) while the feature count is minimized. This enforces an explicit parsimony-accuracy operating point before entering the class-level search, ensuring that downstream conclusions reflect necessary information rather than incidental redundancy.

The method 600 begins with block 602, in which the computing device 102 performs coarse feature refinement with iterative SHAP distillation. This is Phase I of the illustrative multi-phase framework, and includes iterative descriptor distillation driven by SHAP importance and SHAP interaction structure. Starting from all descriptors (e.g., K=895 descriptors), a supervised model (e.g., random forest by default; extra trees interchangeable) is trained and evaluated with scaffold-aware folds on a designated split (Train-Test for training artifacts, Validation for reported metrics). After each iteration t, SHAP feature importance Sᵢ and pairwise SHAP interactions φᵢⱼ are computed. Here, i and j are feature indices. The next iteration retains: (i) the smallest set of descriptors whose cumulative SHAP importance reaches 75% of the total mass; and (ii) for each retained descriptor, the top 25% interaction partners by |φᵢⱼ| to preserve synergistic structure. This learn→explain→prune loop runs for a predetermined number of iterations, such as 30 iterations. Across iterations, the predictive sufficiency curve (F1±fold SD vs. fraction of features kept) identifies the efficiency knee. In the illustrative embodiment, Iteration 15 (t=15) provided the best/stable F1 with a severe feature reduction (˜70 survivors), and low fold-to-fold variance.

In block 604, the computing device 102 performs fine feature refinement with exhaustive model training and evaluation for feature classes. This is Phase II of the illustrative multi-phase framework, and includes an exhaustive search over a reduced number of class combinations as compared to an exhaustive search over features (e.g., 2¹³ rather than 2⁸⁹⁵) and further identifies minimal indistinguishable class sets via statistical post-processing. The retained features from the best/stable iteration of Phase I (e.g., ˜70 survivors from Iteration 15 in the illustrative embodiment) are mapped into a number of chemically interpretable feature classes (e.g., 13 classes including Molar Refractivity, Polarity, Composition, Size, Mass, Shape, Topology, Charge, Lipophilicity, plus others such as Electrotopological state, Fingerprint Density, Functional Groups, Elemental Counts, Electronic Counts, Ring Saturation in the illustrative embodiment). Models are trained and evaluated on every non-empty subset of classes (e.g., 2¹³ combinations). For each combination, F1 is recorded; the set of Pareto-efficient points (non-dominated in F1 vs. number of classes) traces the accuracy-compactness trade-off A post-processing step designates a combination “indistinguishable” if its F1 Score is within TAU=0.01 of the global best; from these, minimal indistinguishable sets are extracted by removing any supersets that do not improve beyond TAU.

To quantify stability and mechanism, Phase II also computes class frequencies across minimal sets, co-occurrence statistics for pairs (and optionally higher-order intersections), and drop-one ablations within top minimal sets to measure ΔF1 when each class is removed. A Jaccard similarity analysis over minimal sets yields an “equivalence map” that clusters families of minimal recipes, revealing exchangeable vs. backbone classes. These analyses generate visual artifacts (e.g., word clouds, co-occurrence matrices/word-pairs, ablation bars, Pareto curve, equivalence heatmap) that summarize structure without overwhelming with text.

In block 606, the computing device 102 performs its finest feature refinement using features within minimal class sets. This is Phase III of the illustrative multi-phase framework, and confines any remaining feature-level combinatorics to a drastically smaller subset as compared to the full set of K features (e.g., K=895). Accordingly, rather than exhaustively testing features across the full 2^{K} (e.g., 2⁸⁹⁵) space, Phase III limits the search to descriptors contained within the minimal class sets uncovered in Phase II. This focuses compute on a small, high-yield frontier, allowing practical feature-subset experiments (e.g., tens to hundreds or low thousands instead of astronomically many), and enables direct mapping from minimal class sets to minimal feature sets that remain interpretable and mechanism-consistent. After performing feature refinement the method 600 is completed. One or more LNP QSAR models 210 may be trained using the refined input feature set as determined by the method 600, which may improve prediction and training performance and/or efficiency as described above.

### Examples

In an experiment, training data was collected from an evaluation of existing literature. As described above, the training data includes chemical formulation information for each LNP in the study, along with associated measured target variables from in vitro experiments. In the illustrative experiment, 21 independent studies were utilized to collect data for a total of 6,454 LNP formulations. For each LNP, ChemDraw v22.2.0 was used to draw the chemical structure of the constituent molecules and to obtain their simplified molecular-input line-entry system (SMILES) strings. WebPlotDigitizer44 and manual data curation on published figures was used to collect the values of the target variables for each LNP. Numeric target values (e.g., luciferase expression values in relative light units, RLUs) underwent log-transformation as described above. The log transformation was applied prior to normalization to reduce skewness and stabilize variance in the distribution of target values, thereby ensuring more consistent scaling across datasets with differing measurement ranges. This log transformation step was followed by min-max normalization within each dataset as described above.

The complete in vitro dataset for ML model development was divided into three distinct subsets: (i) Hyperparameter tuning set containing 1,512 LNP formulations (Data14), exclusively used for optimizing ML model parameters, (ii) Model training and testing set containing 4,849 LNP formulations (Data1 through Data13), and (iii) an additional dataset from seven independent studies (Data15 through Data21), thus creating an entirely independent validation dataset. The model training and testing dataset was further partitioned using scaffold-based 5-fold cross-validation. Specifically, Bemis-Murcko molecular scaffolds were generated from SMILES representation of the ionizable lipid of each LNP formulation, and those scaffolds were used to perform five splits via Scikit-learn's GroupKFold method. To quantitatively assess molecular distinctness, the mean Tanimoto similarity scores were calculated between ionizable lipids in the testing set and those in the training set within each fold. For this, each ionizable lipid was represented using Morgan fingerprints with a radius of 2. Pairwise Tanimoto similarity scores were then computed between each testing-set lipid and all training-set lipids, and the mean similarity was calculated for each testing-set lipid. Tanimoto similarity scores generally ranged between 0.10 and 0.25, indicating low structural overlap and thus validating the scaffold-based splitting approach.

In the illustrative experiment, 11 diverse molecular featurizers were used, as shown in Table 1. To further enrich the vectors generated by each featurizer, seven additional composition-level features were appended: the molar ratios of each of the four LNP constituents, the encapsulated nucleic acid type (0 for mRNA, 1 for siRNA), the lipid-to-nucleic acid ratio, and the drug dosage. Repeating this process for all eleven featurization methods resulted in eleven distinct sets of numerical features for each LNP, enabling comprehensive evaluation across multiple featurization strategies.

TABLE 1
Illustrative featurizer techniques.

| Featurizer | |
|---|---|
| ID | Featurizer Name |
| 1 | 2D Descriptors |
| 2 | Normalized descriptors |
| 3 | 3D descriptors |
| 4 | Daylight fingerprints |
| 5 | Atom pair fingerprints |
| 6 | Morgan fingerprints |
| 7 | PyTorch graph |
| 8 | WeaveNet graph |
| 9 | DMPNN graph |
| 10 | Descriptors + PyTorch graph |
| 11 | Daylight fingerprints + PyTorch graph |

Regarding the illustrative featurization strategies, RDKit 2D descriptors are a set of numerical features that quantitatively characterize various physicochemical, topological, and structural properties of molecules based solely on their two-dimensional (2D) representations. These descriptors are computed from the molecular graph—defined by atoms and bonds—and capture a wide range of molecular properties, including molecular weight, lipophilicity (e.g., Log P), topological polar surface area (TPSA), hydrogen bond donor and acceptor counts, partial atomic charges, rotatable bond count, and various electronic and geometric indices. In addition, RDKit provides specialized descriptor families such as BCUT2D descriptors, Kappa shape indices, EState indices, and VSA (van der Waals surface area) descriptors, each offering unique insights into aspects like molecular complexity, atomic environments, and charge distribution. These descriptors are deterministic, interpretable, and computationally efficient to calculate, making them particularly well-suited for use in cheminformatics and machine learning applications. In the illustrative experiment, RDKit 2D descriptors were used to generate comprehensive feature vectors that capture the diverse physicochemical properties of lipid nanoparticle constituents, enabling robust modeling of structure-activity relationships.

Descriptastorus normalized descriptors are an extended set of molecular descriptors generated using the Descriptastorus package, which builds upon RDKit's descriptor set and provides additional preprocessing capabilities, including feature scaling and normalization. These descriptors encompass a wide range of physicochemical, topological, and electronic properties, similar to RDKit 2D descriptors, but are preprocessed to ensure that all features are on comparable numerical scales—typically through min-max normalization or z-score standardization. Normalizing descriptors helps mitigate the effects of differing value ranges across features, which can improve convergence and performance of many machine learning algorithms. In the illustrative experiment, Descriptastorus normalized descriptors were used to generate standardized feature vectors for each LNP constituent, allowing for more balanced and interpretable input to the ML models and facilitating consistent feature importance analysis across different descriptors.

RDKit 3D descriptors are a set of molecular features that quantify the three-dimensional geometric and spatial properties of molecules, derived from their conformer-based 3D structures. These descriptors capture aspects such as molecular shape, size, symmetry, and spatial distribution of atoms, which are critical for understanding molecular behavior in a biological environment. Commonly computed 3D descriptors include measures like asphericity, eccentricity, spherocity index, principal moments of inertia (PMI), and the plane of best fit (PBF). These features help characterize the overall spatial arrangement and anisotropy of molecules, which can influence properties such as nanoparticle assembly, membrane interactions, and biological uptake. In the illustrative experiment, 3D descriptors calculated using RDKit were used to complement 2D features, providing additional geometric insights into the LNP constituents for more comprehensive machine learning-based structure-activity modeling.

Daylight fingerprints, as implemented through the RDKitFPGenerator in RDKit, are a widely used molecular representation that encodes structural information based on the presence of substructures within a molecule. These fingerprints are generated by identifying all possible linear paths of atoms up to a specified length (typically between 1 and 7 bonds) within a molecular graph. Each identified path is hashed into a bit in a fixed-length binary vector, where each bit position corresponds to a specific molecular fragment or substructure. The result is a high-dimensional binary fingerprint in which a bit value of 1 indicates the presence of a particular path or pattern in the molecule, and a value of 0 indicates its absence. This representation is particularly effective for capturing structural features such as rings, chains, and functional groups. In the illustrative experiment, RDKit was used to generate daylight fingerprints using the following parameters: radius=2 which represents the fingerprint radius, size=1024 which represents the length of the fingerprint vector, chiral=False which ignores chirality in fingerprint generation, and bonds=True which considers bond order in fingerprint generation.

RDKit's Atom Pair fingerprints are a type of molecular representation that captures the topological relationships between pairs of atoms within a molecule. These fingerprints are generated by identifying all atom pairs in a molecule and encoding their atom types and the shortest path distance (number of bonds) between them. Each atom pair is then hashed into a fixed-length binary or count-based fingerprint vector. This representation effectively captures both local and distal structural features, making it suitable for distinguishing molecules based on their overall shape and connectivity patterns. Atom Pair fingerprints are particularly useful for tasks involving molecular similarity, virtual screening, and structure-activity relationship modeling. In the illustrative experiment, Atom Pair fingerprints, with size=1024, generated using RDKit, were used as one of the featurization strategies to encode the structural topology of LNP constituents for downstream machine learning applications.

Morgan fingerprints, also known as circular fingerprints, are a widely used molecular representation based on the extended-connectivity fingerprint (ECFP) algorithm. Generated using RDKit's implementation, these fingerprints encode the local atomic environments around each atom by iteratively hashing circular substructures up to a specified radius (e.g., radius=2). The resulting structural patterns are hashed into a fixed-length binary vector, where each bit represents the presence or absence of a particular substructure. Morgan fingerprints are especially effective for capturing local chemical environments and substructural diversity, making them well-suited for molecular similarity comparisons and machine learning applications. In the illustrative experiment, Morgan fingerprints of size=1024 were used to represent the structural characteristics of LNP constituents in a compact and computationally efficient form.

PyTorch Geometric is a library that provides tools for deep learning on graph-structured data, including molecular graphs. It enables the construction and training of GNNs that can learn complex patterns from molecular graphs where atoms are nodes and bonds are edges. This framework is particularly well-suited for capturing both the local and global structural information in molecules, making it highly effective for various cheminformatics tasks, such as molecular property prediction, reaction outcome prediction, and molecular docking.

The MolGraphConv featurizer from DeepChem encodes molecules as graph-based representations, where atoms are treated as nodes and bonds as edges in a molecular graph. This featurizer generates rich atomic-level features such as atom type, formal charge, hybridization, aromaticity, and more, along with bond features like bond type and conjugation. These graphs serve as input to graph convolutional neural networks (GCNs), which learn to propagate and integrate information across local neighborhoods in the graph. The MolGraphConv featurizer thus enables models to learn context-aware structural patterns directly from molecular graphs. In the illustrative experiment, the MolGraphConv featurizer was employed to generate graph-based molecular representations for LNP constituents, enabling the application of GCNs to predict LNP activity and cytotoxicity.

The Directed Message Passing Neural Network (DMPNN) featurizer, implemented in DeepChem, provides a graph-based representation tailored for use with message passing neural networks. Unlike standard GCNs, DMPNN explicitly models directed messages between bonds rather than atoms, allowing for directional flow of chemical information across the molecular graph. This approach captures more detailed chemical context and bond-level interactions, which can be crucial for understanding reaction mechanisms and molecular properties. The DMPNN featurizer constructs bond-centered graph features that are particularly well-suited for deep learning architectures that operate on molecular graphs. In the illustrative experiment, DMPNN featurization was used to extract enriched graph features for LNP constituents, facilitating predictive modeling through advanced graph-based neural networks.

To capture both structural connectivity and physicochemical properties of lipid nanoparticle constituents, a hybrid featurization strategy was developed by combining PyTorch Geometric-based molecular graphs with RDKit 2D descriptors. The graph-based features were constructed using PyTorch Geometric, where atoms and bonds were represented as graph nodes and edges, respectively, and included rich atom-level and bond-level attributes. These graph representations allow machine learning models to capture local atomic environments and molecular topology through graph-based deep learning architectures such as GCNs and GATs. To complement these structural features, RDKit 2D descriptors were concatenated with each molecular graph's global feature vector, adding interpretable information such as lipophilicity, polar surface area, molecular complexity, partial charges, and hydrogen bonding potential. This integrated hybrid representation combines the advantages of data-driven graph learning with interpretable, handcrafted molecular descriptors, thereby enhancing model performance and interpretability in structure-activity prediction tasks.

Illustratively, another set of hybrid features was constructed by combining PyTorch Geometric molecular graphs with Daylight-style fingerprints, generated using RDKitFPGenerator. The graph-based component captured detailed structural information through node and edge features, while the Daylight fingerprints provided a binary vector encoding the presence or absence of predefined substructures and molecular fragments. These fingerprints offer a compact summary of structural motifs and are widely used for similarity-based learning tasks. By integrating these binary fingerprint vectors into the global features of the molecular graphs, the hybrid representation enables simultaneous learning from both detailed graph connectivity and high-level structural patterns, thereby offering a more comprehensive molecular characterization for predictive modeling.

Six machine learning models, as shown in Table 2, were trained using the input features. Accordingly, the machine learning models used in the illustrative experiment included balanced random forest (BRF), extra trees, gradient boosting, support vector machine (SVM), graph convolutional network, and graph attention network.

TABLE 2
Illustrative machine learning models.

| ML | |
|---|---|
| Model ID | ML Model Name |
| 1 | Balanced random forest (BRF) |
| 2 | Extra trees |
| 3 | Gradient boosting |
| 4 | Support vector machine (SVM) |
| 5 | Graph convolutional network |
| 6 | Graph attention network |

The initial step for training involved hyperparameter tuning, utilizing a dedicated dataset specifically curated for this purpose. Hyperparameters for each of the six ML algorithms listed in Table 2 were systematically optimized, ensuring the models were finely tuned to maximize predictive accuracy and generalizability. In the illustrative experiment, for balanced random forest, n_estimators=100, max_depth=10, min_samples_leaf=4, min_samples_split=10 were selected as final parameter values for scaffold-based five-fold cross-validation. n_estimators=50, max_depth=None, min_samples_split=10, min_samples_leaf=4 were selected as final parameter values for the extra trees classifier. n_estimators=100, max_depth=3, learning_rate=0.01 were selected as the final parameter values for gradient boosting classifier. C=1.0, max_iter=200 were selected as the final parameter values for the support vector classifier with a linear kernel. hidden_dim=32, learning_rate=0.001, epochs=200 were selected as the final parameter values for graph convolutional network-based classifier. hidden_dim=64, num_heads=4, learning_rate=0.01, epochs=200 were selected as the final parameter values for graph attention network-based classifier. Training proceeded for each of the six models using the training and test data set including 4,849 LNP formulations as described above.

Model performance of each of the six trained models was assessed using multiple metrics suitable for both binary and multiclass classification tasks. Specifically, for each fold and classification task, accuracy, precision, recall, F1 score, Matthews correlation coefficient (MCC), ROC-AUC score, and Cohen's kappa coefficient were computed for each individual class. Class-weighted metrics were then calculated to account for the class imbalance by weighting each metric by the number of LNP formulations per class. These class-weighted metrics were averaged across the five folds to derive a reliable and robust measure of model performance.

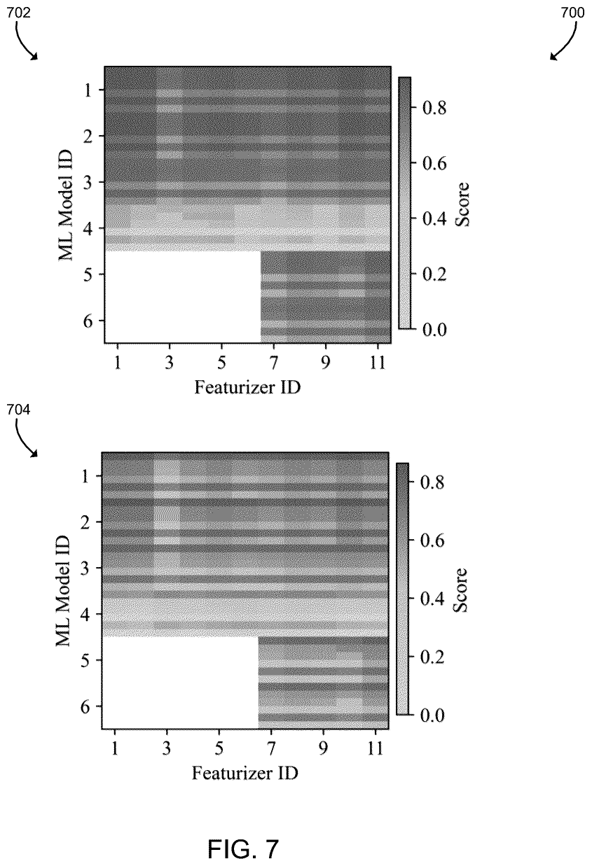

Referring now to FIG. 7, diagram 700 illustrates heatmaps summarizing model performance for binary classification and multiclass classification of LNPs based on activity using various ML model-featurizer combinations. Chart 702 illustrates binary classification results, and chart 704 illustrates multiclass classification results. In the charts 702, 704, each rectangular block corresponds to a unique model-featurizer pair, and was further subdivided into six horizontal segments, from top to bottom, representing class-weighted average.

For binary classification of LNPs based on activity, as shown in chart 702, the illustrative system 100 achieved high predictive accuracy across several model-featurizer combinations. Among all models, balanced random forest (BRF) and extra trees (ET) algorithms (ML model IDs 1 and 2) consistently yielded the best performance across most featurization techniques. Similarly, among the featurizers, molecular descriptors and normalized descriptors (Featurizer IDs 1 and 2) outperformed other representations, yielding classification accuracy scores above 90%, with consistently high precision, F1 scores, and MCC values. For example, with input features generated using RDKit descriptors, the BRF achieved an accuracy of 90.7%, while ET yielded 90.4% accuracy. In contrast, graph-based models such as graph convolutional network (GCN) and graph attention network (GAT) (ML model IDs 5 and 6), when paired with graph-based representations such as PyTorch and MolGraphConv (Featurizer IDs 7 and 8), resulted in slightly lower accuracy ranges, typically between 77% and 83%. SVM with a linear kernel (ML model ID 4) exhibited the poorest performance, often failing to exceed 50% accuracy, indicating limited ability to capture complex feature-activity relationships in this task.

In the case of multiclass classification of LNP activity, as shown in the chart 704, a similar trend was observed, though overall performance across all models and featurization techniques was slightly lower compared to binary classification due to increased classification complexity. BRF and ET continued to be the top-performing models with accuracy scores of ˜86% when paired with molecular descriptors, while graph-based models achieved accuracy scores up to ˜81%.

For binary classification based on cell viability (not shown), the results demonstrated strong and consistently high performance across all ML models. In the illustrative experiment most model-featurizer combinations achieved accuracy and F1 scores in the range of 90-91%, indicating that the classification of LNPs based on cell viability was comparatively easier and more robust across diverse modeling approaches. Descriptor-based features continued to yield the highest performance overall, but graph-based representations also performed competitively in this task.

Across all tasks, the best results were consistently achieved when ensemble-based models were paired with descriptor-based featurization techniques. These combinations leveraged the strengths of both components: ensemble models were adept at capturing non-linear relationships, handling high-dimensional and heterogeneous feature spaces, and modeling complex interactions among molecular and formulation-level attributes, while descriptor-based features provided interpretable and chemically meaningful representations of molecular properties relevant to LNP performance. In contrast, graph-based models, though theoretically capable of capturing intricate structural relationships through message passing, showed more variable performance depending on the task. While they performed competitively in cell viability classification, their performance in activity classification was relatively lower. This suggested that structure-toxicity relationships may be more strongly influenced by local atomic environments or substructural motifs, which are well captured by graph representations, whereas LNP activity appeared to depend more heavily on global physicochemical properties, which were better represented by descriptors.

After completing cross-validation, the models' predictive generalizability was assessed by applying the trained models to an independent dataset comprising entirely unseen LNP formulations. This evaluation step provided validation of the models' capabilities to generalize beyond their training data, thus reflecting their potential applicability to novel formulations encountered in practical scenarios.

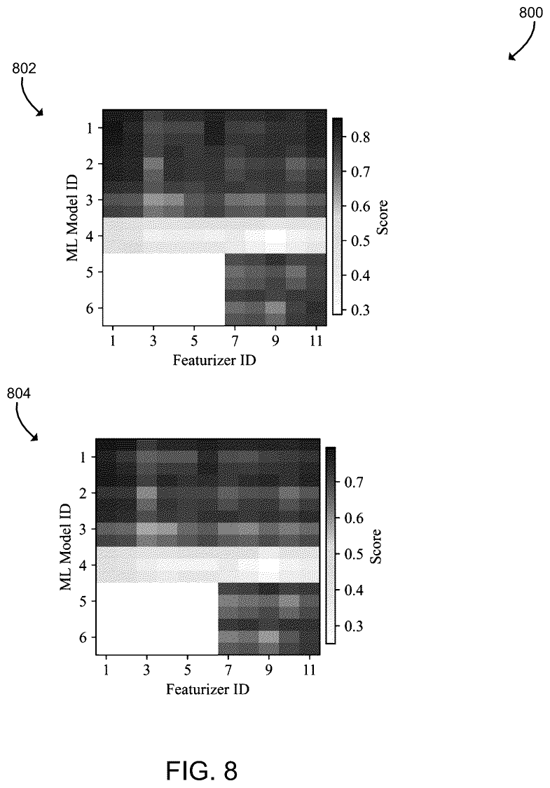

As described above, to evaluate the generalizability of the trained ML models, the models' performance was tested on an independent validation dataset comprising LNP formulations curated from seven separate studies that were not used during model training, testing, or hyperparameter tuning. Referring now to FIG. 8, diagram 800 illustrates heatmaps summarizing model performance for binary classification and multiclass classification of LNPs based on activity, evaluated on an independent external validation dataset (e.g., Data15 through Data21). Chart 802 summarizes model performance for binary classification, and chart 804 summarizes model performance for multiclass classification. In the charts 802, 804, each rectangular block corresponds to a unique model-featurizer pair and was subdivided into three horizontal segments, from top to bottom, representing class-weighted average values (over five-fold scaffold-based cross-validation) of accuracy, precision, and F1 score, respectively.

As shown in the diagram 800, the overall accuracy, precision, and F1 scores on the independent dataset were slightly lower compared to the scaffold-based five-fold cross-validation results, which is expected when models are challenged with completely unseen molecular scaffolds and formulation spaces. Nonetheless, several model-featurizer combinations maintained robust predictive performance, with many achieving accuracy and F1 scores of ˜84% for binary classification and ˜79% for multiclass classification, indicating a strong degree of model generalization. For example, the BRF model paired with molecular descriptors achieved accuracy and F1 scores of 83.9% and 84.6%, respectively, for the binary classification task, and accuracy and F1 scores of 79.6% and 78.1%, respectively, for the multiclass classification task.

Consistent with trends observed in the primary dataset, descriptor-based features paired with ensemble-based models continued to yield the highest overall performance. Other model-featurizer combinations, including those involving graph-based features and gradient boosting, also showed moderately strong generalization in both classification tasks. These findings reinforced that model-featurizer combinations that captured a broad spectrum of physicochemical and formulation-level features were more likely to generalize well to novel LNP formulations, thereby highlighting the potential of such ML frameworks for real-world screening and design of next-generation LNPs.

To gain a deeper understanding of which molecular and compositional features most strongly influence LNP activity, feature importance and interaction analysis was performed using SHAP47,48 (Shapley additive explanations). This analysis was conducted on features generated using molecular descriptors for all LNP constituents, namely, ionizable lipid, helper lipid, cholesterol, and PEG-lipid, along with additional compositional features, including the molar ratios of each constituent, the type of encapsulated nucleic acid, the lipid-to-nucleic acid ratio, and the drug dosage.

A BRF binary classification model was trained using the complete set of molecular and compositional features. SHAP values were then computed to quantify the contribution of each feature to the model's prediction of high LNP activity. In this framework, SHAP values represent the marginal contribution of a feature to a model's output, computed by taking a weighted average over all possible feature coalitions. For tree-based models such as RF, SHAP values were computed using a TreeSHAP algorithm, which analytically derives exact Shapley values based on the structure of the decision trees.

For each feature i, feature importance, Sᵢ, was determined by calculating the average of the absolute SHAP values for the feature across all LNP samples. This average absolute SHAP value represents the overall impact of that feature on model predictions, regardless of the direction of its effect (positive or negative). Features were then ranked in descending order of importance based on these values.

To evaluate whether the most important features also performed well in isolation, separate BRF models were trained using only one feature at a time. Each model was evaluated using five-fold cross-validation to compute performance metrics such as accuracy, precision, recall, and F1 score. This allowed the standalone predictive power of individual features to be assessed, independent of feature interactions.

Given that molecular features often exhibit complex, non-additive interactions, the analysis the extended to examine feature interactions using SHAP interaction values. These values decompose the total SHAP value into main effects and pairwise interaction effects, thereby quantifying how the presence of one feature modifies the contribution of another to the prediction. For each pair of features (i,j), SHAP interaction values were computed by comparing the SHAP value of a feature when the other feature is present versus when it is absent, symmetrically across all permutations. The interaction importance between two features was then defined as the average absolute SHAP interaction value |φ_{i,j}| across all samples, where φ_{i,j} denotes the pairwise interaction term between features i and j.

Building upon the feature importance and interaction analyses, multiple feature clusters were constructed to investigate how combinations of informative and interacting features influence model performance. Clusters were initially formed by selecting the top-N most important features (N=3, 5, 7, and 10), and for each selected feature, identifying the features with which it had the strongest interactions. Additional clusters were created from second- and third-tier features to capture broader interaction patterns within the feature space. For each feature cluster, a weighted interaction importance score was calculated to quantify the collective significance of the group. This score was computed by summing the weighted interaction contributions between all feature pairs in the cluster. Each pairwise contribution was defined as W_{i,j}=(Sᵢ+Sⱼ)×φ_{i,j} where Sᵢ and Sⱼ are the average absolute SHAP importance values for features i and j, respectively. The total interaction importance score for a cluster c was obtained by summing the contributions across all pairs in the group as W_{c}=ΣᵢΣ_{j≠i}W_{i,j}.

To validate the relevance of each feature cluster, BRF classifiers were trained using only the features within each group and model performance using five-fold cross-validation was evaluated. The relationship between the predictive accuracy of each cluster and its corresponding weighted interaction score was then analyzed. This enabled assessment of whether combinations of individually important and highly interactive features translated into improved predictive performance, thereby providing a mechanistic and interpretable understanding of the ML model behavior in the context of LNP design.

To gain insights into the molecular determinants of LNP performance and interpret the predictions of the trained ML models, a comprehensive feature analysis was conducted using SHAP importance and interaction scores. This SHAP-based feature importance analysis revealed that several of the most influential features corresponded to formulation-level parameters and fundamental physicochemical properties of the LNP constituents. Among the most prominent were features related to the electrostatic characteristics of the ionizable lipid, particularly descriptors capturing the magnitude and distribution of partial atomic charges, which play a key role in nucleic acid complexation, membrane interaction, and endosomal escape. Formulation-level variables such as the ratio of lipid to RNA and LNP dosage emerged as critical predictors, highlighting the importance of composition and dosing strategy in determining LNP activity. Several highly ranked descriptors also captured molecular shape and topological complexity, including features that quantify molecular branching, rigidity, and spatial asymmetry, which are known to influence nanoparticle self-assembly, stability, and endosomal escape. Other important features represented surface charge distribution, hydrophobic/hydrophilic surface distribution, and molecular volume, all of which can modulate self-assembly, stability, cellular uptake, biodistribution, and interactions with biological membranes. These findings underscored that LNP activity was a multifactorial phenomenon, shaped by a combination of electrostatics, lipophilicity, topology, shape, and formulation design parameters.

SHAP-layered violin plots (not shown) provided deeper insights into how specific molecular and formulation-level features influenced model predictions of LNP activity. For 2D descriptors, features such as the maximum partial charge on the ionizable lipid showed a strong positive association with high LNP activity. Namely, higher values of this descriptor consistently corresponded to higher SHAP values, indicating that an increase in surface charge enhanced the likelihood of an LNP being classified as “high” activity. Similarly, lipid-to-RNA ratio showed a strong negative association with LNP activity. Namely, lower ratios (more nucleic acid content) positively contributed to high activity classification, while very high ratios reduced the probability of high activity. A similar inverse relationship was observed for molecular shape complexity, where lower values, typically associated with more compact molecular shapes, aligned with increased activity predictions, while highly complex or branched molecules showed diminished predictive contributions. Interestingly, LNP dosage exhibited a bell-shaped relationship, with moderate dosage levels being most favorable for high activity classification, whereas very high dosages appeared to reduce the predicted probability of success, possibly reflecting saturation effects or cytotoxicity concerns. Other descriptors also demonstrated directional patterns in their influence, such as molar refractivity and partial charge (calculated as sum of the atomic van der Waals surface area contributions of each atom), with the former showing higher SHAP values at low descriptor values, while the latter contributed positively when its values were high, emphasizing the role of localized charge distribution on the ionizable lipid in mediating LNP interactions with biological membranes.

A similar interpretation emerged from violin plots for 3D descriptors, where lipid-to-RNA ratio and LNP dosage again followed trends consistent with those seen in 2D descriptors. Additional 3D features revealed nuanced structure-activity relationships. For instance, PMI1, a descriptor of molecular spatial distribution and shape elongation, contributed positively to activity when its values were moderate to slightly high, while very low values were linked to reduced activity predictions. The molar ratio of the ionizable lipid, a key formulation-level feature, also showed a strong positive correlation with activity. Namely, higher proportions of the ionizable lipid increased SHAP values, suggesting their crucial role in effective nucleic acid complexation and delivery. Together, these patterns highlighted that both molecular-level properties (e.g., charge distribution, shape, surface area) and formulation-level parameters (e.g., lipid ratios, dosage) shaped the overall biological performance of LNPs.

To investigate whether the most important features also exhibited high standalone predictive power, separate BRF models were trained using only one descriptor at a time and evaluated their performance using five-fold cross-validation. For this investigation, model accuracy plotted against SHAP importance scores revealed no clear monotonic trend. While some of the top-ranked features did achieve relatively high accuracies in isolation, many others did not. This observation suggested that model performance was not governed solely by the contribution of individual features but was instead heavily influenced by complex interactions among multiple features.

To further investigate this, the SHAP interaction values between features was analyzed. This analysis revealed that many top-ranked features exhibited substantial interactions with a broad set of other descriptors. Several of the strongest feature interactions occurred between descriptors that captured complementary physicochemical attributes. For example, features reflecting the molecular polarizability and hydrophobic surface area exhibited strong interactions with descriptors characterizing electronic charge distribution, such as partial atomic charges. Similarly, descriptors related to the molecular topology and shape demonstrated pronounced interactions with features capturing the electrostatic potential and local electronic environments. These interactions underscored the non-additive nature of the feature space, where the predictive contribution of a feature was often modulated by the presence or absence of other interacting features. Collectively, these results reinforced the idea that high model performance emerged from the synergistic effects of multiple interacting molecular descriptors rather than isolated contributions of individual features.

To further validate the significance of both individual feature importance and inter-feature dependencies, model performance was evaluated using distinct clusters of features, each defined based on their individual SHAP importance and their strongest interaction partners. A clear monotonic relationship was observed between the model accuracy and the total interaction importance of each cluster, quantified by aggregating the weighted interaction contributions across all feature pairs within a group. This trend reinforced the idea that model performance was not dictated solely by individual influential features, but rather by synergistic interactions among multiple complementary features. Importantly, this approach allowed identification of feature combinations that, together, contributed to significantly improved predictive accuracy, even when individual features within the group were not the highest-ranking by importance in isolation.

A closer inspection of the best-performing feature clusters revealed that these groups predominantly comprised descriptors capturing complementary and diverse physicochemical attributes. For example, clusters enriched in features related to partial atomic charges and electronic properties were often paired with descriptors reflecting molecular shape, lipophilicity, and formulation-level parameters such as lipid-to-RNA ratio and LNP dosage. Other clusters combined topological indices with electrostatic surface area descriptors and molecular branching characteristics, capturing both structural complexity and surface interactions. The consistent presence of formulation-specific variables, including lipid to RNA ratio and dosage, alongside atomic-level descriptors, highlighted the importance of integrating both molecular and formulation features for accurate prediction of LNP activity. These findings provided compelling evidence that groups of chemically and biophysically relevant features, when selected based on both importance and interaction, formed a more informative and predictive representation of LNPs than individual features alone.

In another experiment, to assess the applicability of the developed ML framework beyond in vitro predictions, model performance was evaluated on an independent in vivo dataset including 187 LNP formulations using a transfer learning approach. In this experiment it was shown that, although the accuracy, precision, and F1 scores were slightly lower than those observed for in vitro classification, the overall performance for in vivo prediction remained strong. For instance, BRF paired with descriptor-based features achieved over 82% accuracy, precision, and F1 score, indicating that despite domain shifts between in vitro and in vivo conditions, the knowledge transferred from the in vitro-trained models retained significant predictive value in the in vivo context, even with limited data availability.

The reduction in performance compared to in vitro models can be attributed to several factors. First, the in vivo dataset was considerably smaller, comprising only 187 LNP formulations, which limited the model's ability to learn generalizable patterns during fine-tuning. Second, predicting in vivo biological behavior is inherently more complex and multifactorial than in vitro classification tasks. In vivo outcomes are influenced by a multitude of additional biological and systemic parameters such as tissue penetration, immune clearance, and pharmacokinetics/pharmacodynamics. Third, the experimental in vivo dataset likely suffered from class imbalance, a common challenge in biological datasets, particularly in studies where only the most promising formulations from in vitro screens were taken forward for in vivo testing, leading to an overrepresentation of high-activity formulations.

To mitigate this class imbalance, data augmentation strategies were implemented using borderline SMOTE and ADASYN. Both of those oversampling techniques led to noticeable improvements in model performance, particularly for poorly performing model-featurizer combinations. For example, the accuracy of the extra trees model with daylight fingerprint features increased from 65.6% to 75.4% after applying borderline SMOTE and further improved to 78.5% with ADASYN. These improvements were consistent across other evaluation metrics as well, highlighting the importance of addressing data imbalance in enhancing model generalizability. Together, these results demonstrated that even under data-limited and complex biological conditions, transfer learning strategies, when combined with appropriate augmentation techniques, may perform predictive modeling of in vivo nanoparticle performance.

To summarize, in the illustrative experiment, a machine learning framework was developed to predict the biological performance of lipid nanoparticles based on their molecular and formulation-level features. Using an extensive in vitro dataset comprising over 6,400 LNP formulations, a diverse array of featurization strategies were implemented, including molecular descriptors, fingerprints, graph-based representations, and hybrid approaches, combined with multiple machine learning algorithms to classify LNP activity and cell viability. The results of the experiment demonstrated that tree-based ensemble models, particularly balanced random forest and extra trees, paired with descriptor-based features consistently yielded the highest predictive performance, achieving accuracies exceeding 90% in both activity and cytotoxicity classification tasks. Through detailed SHAP-based feature attribution analysis, several physicochemical properties were identified, such as partial charge distribution, molecular topology, lipid-to-RNA ratio, and LNP dosage, as key contributors to model performance. Further analysis revealed that complex interactions among features played a critical role in driving predictive accuracy, underscoring the importance of considering both individual feature relevance and feature interdependencies. Transfer learning to a smaller in vivo dataset, while yielding slightly lower performance due to dataset size limitations and biological complexity, still achieved over 82% accuracy, highlighting the potential of leveraging in vitro-trained models to inform in vivo behavior.

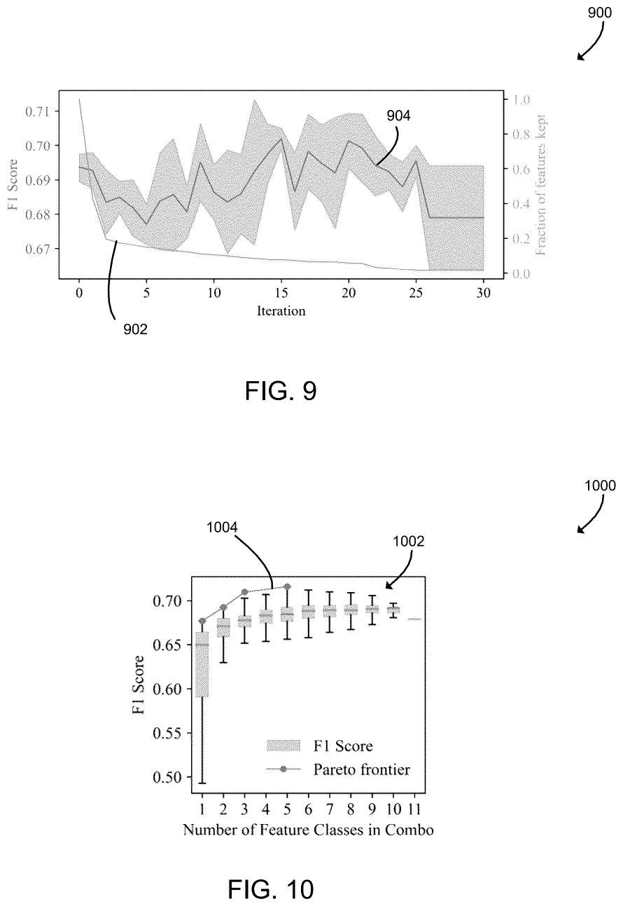

In an experiment, multi-phase feature refinement was performed as described above in connection with FIG. 6. In the experiment, the coarse-refining loop of Phase I compressed the descriptor set from 895 to ˜70 by Iteration 15 while maintaining Validation F1 and displaying narrow fold variance, demonstrating that most original descriptors were redundant for prediction when guided by SHAP importance and interaction structure. Referring now to FIG. 9, diagram 900 illustrates predictive sufficiency across iteration. Diagram 900 is a dual-axis plot of F1 score (left y-axis) and fraction of features retained (right y-axis) versus iteration. As shown, performance remains stable while descriptor set is aggressively distilled, revealing the efficiency knee. The dual-axis sufficiency plot 900 shows a sharp drop in fraction of features kept (curve 902) with a steady F1 trajectory (curve 904±standard deviation), providing a clear accuracy-parsimony knee for downstream analysis and establishing Iteration 15 as the preferred hand-off point.

Mapping the ˜70 survivors into 13 feature classes during Phase II enabled a complete class-combo sweep (2¹³). Minimal indistinguishable class sets were identified via TAU-based post-processing; notably, more than one minimal set delivered F1 statistically indistinguishable from the best, confirming that equivalent “recipes” exist. Frequency and co-occurrence analyses highlighted a recurring backbone: Molar Refractivity, Polarity, Composition, Size & Mass, Shape & Topology, Charge, and Lipophilicity appear disproportionately often among minimal sets, as visualized in word clouds of feature-class prevalence in minimal sets and of recurrent class pairings in minimal sets. Referring now to FIG. 10, diagram 1000 illustrates accuracy versus compactness at the class level. Box-and-whisker plots 1002 show the distribution of F1 scores for each number of feature classes considered; and curve 1004 overlays the Pareto frontier of non-dominated trade-offs. The Pareto frontier, as shown in the diagram 1000, revealed diminishing returns beyond a small number of classes; in other words, compact class combinations capture nearly all achievable performance.

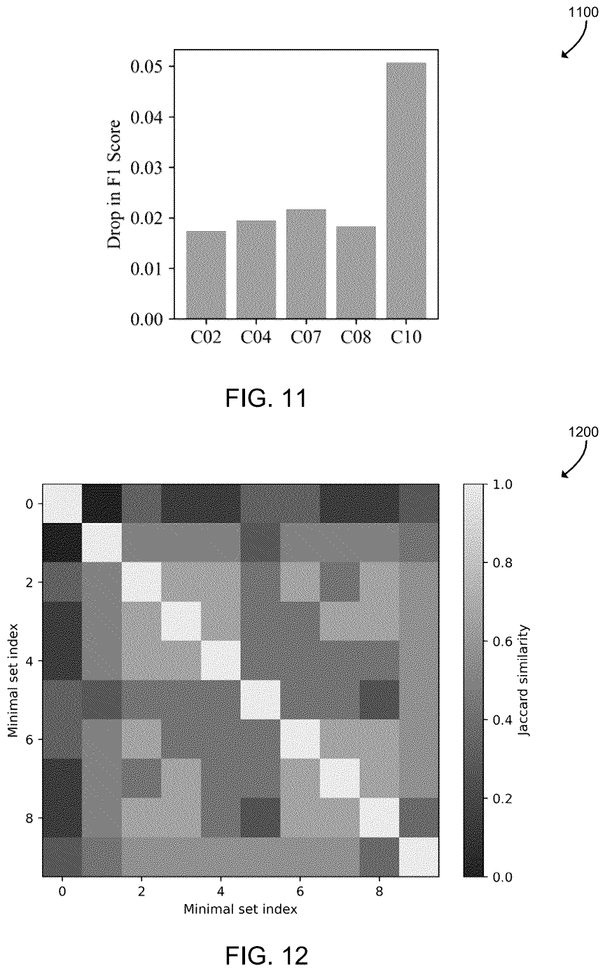

Referring now to FIG. 11, diagram 1100 illustrates drop-one-class ablation within the best recipe. The chart 1100 shows the decrease in F1 when removing each class in turn (C02=Composition; C04=Elemental Counts; C07=Molar Refractivity; C08=Polarity; C10=Size & Mass). Larger drops indicate more indispensable classes. Referring now to FIG. 12, diagram 1200 illustrates equivalence of minimal sets by Jaccard similarity. The diagram 1200 is a heatmap of pairwise overlap between minimal indistinguishable sets. Low similarity confirms that distinct class combinations can achieve near-optimal performance, i.e., multiple viable “recipes” for LNP design.

Accordingly, ablation (drop-one) within top minimal sets quantified indispensability: removing certain backbone classes (e.g., Size & Mass or Molar Refractivity, depending on the recipe) produced the largest ΔF1 penalties, as shown in FIG. 11, while others were partially exchangeable across equivalent sets—consistent with the equivalence map derived from Jaccard similarity, as shown in FIG. 12, which clustered minimal sets into a few families. Together, these outcomes deliver an actionable blueprint: start with an iterative SHAP-guided distillation to a sufficiency knee, perform an exhaustive yet interpretable search across property classes to locate minimal indistinguishable sets, and (optionally) complete a constrained feature-level refinement. The illustrative framework preserved accuracy, exposed mechanism-aligned levers, and converted an intractable 2⁸⁹⁵ problem into a targeted, design-oriented workflow suitable for predictive and generative LNP development.

1. A computing device for quantitative structure-activity relationship model training, the computing device comprising:
a training data manager configured to receive a first training data set comprising a plurality of lipid nanoparticle (LNP) test results, wherein each of the LNP test results comprises an LNP chemical formulation and a corresponding result value for an LNP target variable;
a featurizer configured to extract a plurality of input features for each LNP chemical formulation of the plurality of LNP test results, wherein each of the input features is indicative of an attribute of a component of the LNP chemical formulation or a composition of the LNP chemical formulation; and
a model trainer configured to train a machine learning model to predict the LNP target variable with the plurality of input features and the corresponding result values of the LNP test results.

2. The computing device of claim 1, wherein the LNP target variable comprises LNP activity or cytotoxicity.

3. The computing device of claim 1, wherein each LNP chemical formulation of the LNP test results is indicative of a plurality of components of an LNP, including an ionizable lipid component, a helper lipid component, a PEG-lipid component, a cholesterol component, and a nucleic acid component.

4. The computing device of claim 1, further comprising a data preprocessor configured to:
preprocess the corresponding result values for the LNP target variables of the first training data set to generate processed result values, wherein to preprocess the corresponding result values comprises to (i) log-transform any numeric result values of the corresponding result values to generate log-normalized numeric result values, (ii) normalize the log-transformed numeric result values to generate normalized numeric result values, and (iii) categorize the normalized numeric result values with k predetermined categorical labels using k-means clustering;
wherein to train the machine learning model comprises to train the machine learning model with the plurality of input features and the processed result values.

5. The computing device of claim 1, wherein the machine learning model comprises a decision tree ensemble model.

6. The computing device of claim 5, wherein the decision tree ensemble model comprises a balanced random forest model or an extra trees model.

7. The computing device of claim 1, wherein to extract the plurality of input features for each LNP chemical formulation of the plurality of LNP test results comprises, for a first LNP chemical formulation of the plurality of LNP test results, to:
determine a chemical structure of each component of the first LNP chemical formulation;
extract a plurality of molecular structural features in response to a determination of the chemical structure of the first LNP chemical formulation; and
enrich the plurality of molecular structural features with a plurality of composition-level features associated with the first LNP chemical formulation.

8. The computing device of claim 7, wherein to extract the plurality of molecular structural features comprises to:
encode a plurality of quantitative molecular descriptors into a numerical vector for each component of the first LNP chemical formulation; and
concatenate the numerical vectors for each component of the first LNP chemical formulation.

9. The computing device of claim 7, wherein to enrich the plurality of molecular structural features with the plurality of composition-level features comprises to enrich the plurality of molecular structural features with a molar ratio of a plurality of components of the first LNP chemical formulation, an encapsulated nucleic acid type of the first LNP chemical formulation, a ratio of lipids to nucleic acid of the first LNP chemical formulation, or a drug dosage associated with the first LNP chemical formulation.

10. The computing device of claim 7, wherein to extract the plurality of molecular structural features further comprises to normalize the plurality of molecular structural features.

11. The computing device of claim 1, wherein the model trainer is further configured to perform transfer learning with the machine learning model based on a second trained machine learning model, wherein the first training data set comprises an in vivo data set, and wherein the second trained machine learning model is trained on an in vitro training data set.

12. A method for quantitative structure-activity relationship model training, the method comprising:
receiving, by a computing device, a first training data set comprising a plurality of lipid nanoparticle (LNP) test results, wherein each of the LNP test results comprises an LNP chemical formulation and a corresponding result value for an LNP target variable;
extracting, by the computing device, a plurality of input features for each LNP chemical formulation of the plurality of LNP test results, wherein each of the input features is indicative of an attribute of a component of the LNP chemical formulation or a composition of the LNP chemical formulation; and
training, by the computing device, a machine learning model to predict the LNP target variable using the plurality of input features and the corresponding result values of the LNP test results.

13. The method of claim 12, further comprising:
preprocessing, by the computing device, the corresponding result values for the LNP target variables of the first training data set to generate processed result values, wherein preprocessing the corresponding result values comprises:
log-transforming any numeric result values of the corresponding result values to generate log-normalized numeric result values;
normalizing the log-transformed numeric result values to generate normalized numeric result values; and
categorizing the normalized numeric result values with k predetermined categorical labels using k-means clustering;
wherein training the machine learning model comprises training the machine learning model using the plurality of input features and the processed result values.

14. The method of claim 12, wherein extracting the plurality of input features for each LNP chemical formulation of the plurality of LNP test results comprises, for a first LNP chemical formulation of the plurality of LNP test results:
determining a chemical structure of each component of the first LNP chemical formulation;
extracting a plurality of molecular structural features in response to determining the chemical structure of the first LNP chemical formulation; and
enriching the plurality of molecular structural features with a plurality of composition-level features associated with the first LNP chemical formulation.

15. The method of claim 14, wherein extracting the plurality of molecular structural features comprises:
encoding a plurality of quantitative molecular descriptors into a numerical vector for each component of the first LNP chemical formulation; and
concatenating the numerical vectors for each component of the first LNP chemical formulation.

16. The method of claim 12, further comprising performing, by the computing device, transfer learning with the machine learning model based on a second trained machine learning model, wherein the first training data set comprises an in vivo data set, and wherein the second trained machine learning model is trained on an in vitro training data set.

17. A computing device for quantitative structure-activity relationship estimation, the computing device comprising:
an inference data manager configured to receive a first lipid nanoparticle (LNP) chemical formulation;
a featurizer configured to extract a plurality of input features for the first LNP chemical formulation, wherein each of the input features is indicative of an attribute of a component of the first LNP chemical formulation or a composition of the first LNP chemical formulation; and
a prediction engine configured to predict an LNP target variable result for the first LNP chemical formulation with a trained machine learning model by providing the plurality of input features to the trained machine learning model, wherein the target variable result comprises LNP activity or cytotoxicity.

18. The computing device of claim 17, wherein the trained machine learning model comprises a decision tree ensemble model, wherein the decision tree ensemble model comprises a balanced random forest model or an extra trees model.

19. The computing device of claim 17, wherein to extract the plurality of input features for the first LNP chemical formulation comprises to:
determine a chemical structure of each component of the first LNP chemical formulation;
extract a plurality of molecular structural features in response to determining the chemical structure of the first LNP chemical formulation; and
enrich the plurality of molecular structural features with a plurality of composition-level features associated with the first LNP chemical formulation.

20. The computing device of claim 19, wherein to extract the plurality of molecular structural features comprises to:
encode a plurality of quantitative molecular descriptors into a numerical vector for each component of the first LNP chemical formulation; and
concatenate the numerical vectors for each component of the first LNP chemical formulation.
